(19) United States
(12) Patent Application Publication
(10) Pub. No.: US 2008/0147026 A1
(43) Pub. Date: Jun. 19, 2008

(54) Absorbent fiber with a low absorbent capacity and slow absorption rate

(76) Inventors: Jian Qin, Appleton, WI (US); Richard Norris Dodge, Appleton, WI (US); Peter Robert Elliker, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/640,042

(22) Filed: Dec. 15, 2006

(51) Int. Cl. A61F 13/15

(52) U.S. Cl. 604/358; 8/147

(57) ABSTRACT

An absorbent fiber includes a cellulose fiber that has been modified via chemical modification, where the chemical modification is selected from carboxymethylation, sulfonation, sulfation or phosphonation. The absorbent fiber can be substantially water-insoluble and water-swellable. The absorbent fiber exhibits a Centrifuge Retention Capacity after four hours of between 1.4 and 8 grams per gram and an absorption rate between 0.0001 and 0.01 sec⁻¹. Absorbent articles having a topsheet, a backsheet and an absorbent core can also include the absorbent fiber.

### BACKGROUND

Articles, such as absorbent articles, are useful for absorbing many types of fluids, including fluids secreted or eliminated by the human body. Such articles typically contain an absorbent core that can include superabsorbent materials in a fibrous matrix.

For absorbent articles, U.S. southern pine fluff pulp is used most often and is recognized worldwide as the preferred cellulose fiber for such articles. The preference is based on the fluff pulp's advantageous high fiber length (about 2.8 mm) and its relative ease of processing from a wetlaid pulp sheet to an airlaid web. However, these cellulose fibers typically have a centrifuge retention capacity (CRC) of 1 gram/gram or less and a relatively fast absorption rate of about 0.2 sec⁻¹. For this reason, a cellulose fibrous matrix readily releases acquired liquid on application of pressure. This tendency to release acquired liquid can result in significant skin wetness during use of an absorbent article that includes a core formed exclusively from cellulosic fibers. Such articles also tend to leak acquired liquid because liquid is not effectively retained in such a fibrous absorbent core.

The inclusion of absorbent materials in a fibrous matrix and their incorporation into absorbent articles is known. The incorporation of superabsorbent materials into these products has had the effect of reducing the products' overall bulk while at the same time increasing its liquid absorbent capacity and enhancing skin dryness for the products' wearers.

Superabsorbent materials are generally polymer based and are available in many forms, such as powders, granules, microparticles and films, for example. Upon contact with fluids, such superabsorbents swell by absorbing the fluids into their structures. Superabsorbents are water-swellable, generally water-insoluble absorbent materials having a liquid absorbent capacity of at least about 10, preferably of about 20, and often up to about 100 times their weight in saline.

A variety of materials have been described for use as absorbent materials in absorbent articles. Included among these materials are natural-based materials such as agar, pectin, gums, carboxyalkyl starch and carboxyalkyl cellulose, such as carboxymethyl cellulose. Natural-based materials tend to form gels rather than maintaining a solid form and are therefore not favored in these products. Synthetic materials such as sodium salts of polyacrylates, polyacrylamides, and hydrolyzed polyacrylonitriles have also been used as absorbent materials in absorbent articles. Although natural-based absorbing materials are well known, these materials have not gained wide usage in absorbent articles because of their relatively inferior absorbent properties compared to synthetic absorbent materials, such as sodium polyacrylates. In contrast to the natural-based absorbents, synthetic absorbent materials are generally capable of absorbing large quantities of liquid while maintaining a relatively non-gelatinous form. Synthetic absorbent materials, often referred to as superabsorbent polymers (SAP), have been incorporated into absorbent articles to provide higher absorbency under pressure and higher absorbency per gram of absorbent material. Superabsorbent polymers are generally supplied as particles having a diameter in the range from about 20-800 microns.

In general, superabsorbent materials can quickly absorb fluids insulted into such articles (i.e., a high absorption rate), and have a high capacity which can lock-up fluids immediately. However, this fast absorption rate may also prevent fluid from being distributed to locations away from the fluid insulting point, often referred to as a target zone. This may cause the fluid insulting the target area to reach its capacity much earlier than other regions of the article, resulting in early leakage and low overall product absorbency efficiency. Thus, there is a need for an absorbent article which maintains overall high absorbency properties, but also effectively distributes fluid to regions outside the point of insult to improve overall absorbent efficiency of the article. There is also a need to develop an absorbent material which has a relatively low capacity (i.e., a CRC of less than 10 g/g) and a significantly slow absorption rate (i.e., less than 0.01 sec⁻¹) to obtain these benefits. The present invention seeks to fulfill these needs and provides further related advantages.

### SUMMARY

In response to the needs discussed above, provided is an absorbent fiber which has a relatively low centrifuge retention capacity when compared to conventional superabsorbent material, but a higher centrifuge retention capacity and significantly slower absorption rate when compared to untreated fluff fiber. Also provided is an absorbent article comprising the absorbent fiber of the present invention. Desirably, the absorbent fiber is used in conjunction with superabsorbent material to help absorb extra fluid when the superabsorbent material in a target area of an absorbent article becomes oversaturated. The application of such slow but low capacity absorbent fiber can promote fluid distribution to areas outside of the point of insult to enhance the overall utilization efficiency of the article.

In one particular aspect, an absorbent fiber comprises a cellulose fiber that has been modified via chemical modification, where the chemical modification is selected from carboxymethylation, sulfonation, sulfation or phosphonation, where the absorbent fiber is substantially water-insoluble and water-swellable, and where the absorbent fiber exhibits a Centrifuge Retention Capacity after four hours of between 1.4 and 8 grams per gram and an absorption rate between 0.0001 and 0.01 sec¹.

In some aspects, the absorbent fiber further comprises a crosslinking agent. In some aspects, the absorbent fiber has a degree of substitution of less than 0.5. In other aspects, the absorbent fiber has a degree of substitution of greater than 0.5. In still other aspects, the absorbent fiber has an average degree of carboxyl group substitution of from about 0.1 to about 0.7.

In some aspects, the absorbent fiber further comprises a reagent.

In some aspects, the cellulose fiber is non-regenerated. In other aspects, the cellulose fiber is regenerated. In yet other aspects, the absorbent fiber has the appearance of an unmodified cellulose fiber. In still other aspects, the absorbent fiber has the appearance of a regenerated cellulose fiber.

In a particular aspect, an absorbent article comprises a topsheet, a backsheet and an absorbent core disposed between the topsheet and the backsheet. In this aspect, the absorbent core includes superabsorbent material and absorbent fibers, where the absorbent fibers comprise cellulose fibers that have been modified via chemical modification, where the chemical modification is selected from carboxymethylation, sulfonation, sulfation or phosphonation, and where the modified fibers exhibit a Centrifuge Retention Capacity after four hours of between 1.4 and 8 grams per gram and an absorption rate between 0.0001 and 0.01 sec⁻¹.

In some aspects, the absorbent article further comprises a crosslinking agent. In other aspects, the absorbent article comprises absorbent fibers having a degree of substitution of less than 0.5. In yet other aspects, the absorbent article comprises absorbent fibers having a degree of substitution of greater than 0.5. In still other aspects, the absorbent article comprises absorbent fibers having an average degree of carboxyl group substitution of from about 0.1 to about 0.7.

In some aspects, the absorbent article further comprises a reagent.

In some aspects, the absorbent article comprises cellulose fibers that are non-regenerated. In other aspects, the absorbent article comprises cellulose fibers that are regenerated. In yet other aspects, the absorbent article comprises absorbent fiber having the appearance of unmodified cellulose fibers. In still other aspects, the absorbent article comprises absorbent fiber having the appearance of a regenerated cellulose fiber.

In some aspects, the absorbent article includes an absorbent core which comprises at least about 10% by weight of the absorbent fiber of the present invention. In other aspects, the absorbent article includes an absorbent core which comprises between 10% and 70% by weight of the absorbent fiber of the present invention.

In some aspects, the absorbent article includes an absorbent core which comprises at least about 30% by weight of superabsorbent material. In other aspects, the absorbent article includes an absorbent core which comprises about 60% to about 95% by weight of the superabsorbent material.

In some aspects, the absorbent core further comprises fluff. In other aspects, the absorbent core further comprises a surfactant.

In some aspects, the absorbent article is selected from personal care absorbent articles, health/medical absorbent articles, household/industrial absorbent articles and sports/construction absorbent articles.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1 is a perspective view of one embodiment of an absorbent article that may be made in accordance with the present invention;

FIG. 2 is a plan view of the absorbent article shown in FIG. 1 with the article in an unfastened, unfolded and laid flat condition showing the surface of the article that faces the wearer when worn and with portions cut away to show underlying features;

FIG. 3 is a schematic diagram of one version of a method and apparatus for producing an absorbent core;

FIG. 4A is a cross-section side view of an absorbent bandage of the present invention;

FIG. 4B is a top perspective view of an absorbent bandage of the present invention;

FIG. 5 is a top perspective view of an absorbent bed or furniture liner of the present invention;

FIG. 6 is a perspective view of an absorbent sweatband of the present invention; and

FIG. 7 is a graph of the CRC value as a function of time.

Repeated use of reference characters in the present specification and drawings is intended to represent the same or analogous features or elements of the present invention.

### Test Methods

### Centrifuge Retention Capacity (CRC) Test

The Centrifuge Retention Capacity (CRC) Test measures the ability of the absorbent sample to retain liquid therein after being saturated and subjected to centrifugation under controlled conditions. The resultant retention capacity is stated as grams of liquid retained per gram weight of the sample (g/g). For the fiber samples, the sample to be tested is used as is.

The retention capacity is measured by placing 0.2±0.005 grams of the sample into a water-permeable bag which will contain the sample while allowing a test solution (0.9 weight percent sodium chloride in distilled water) to be freely absorbed by the sample. A heat-sealable tea bag material, such as that available from Dexter Corporation of Windsor Locks, Conn., U.S.A., as model designation 1234T heat sealable filter paper, works well for most applications. The bag is formed by folding a 5-inch by 3-inch (12.7-cm by 7.6-cm) sample of the bag material in half and heat-sealing two of the open edges to form a 2.5-inch by 3-inch (6.4-cm by 7.6-cm) rectangular pouch. The heat seals should be about 0.25 inches (0.64 cm) inside the edge of the material. After the sample is placed in the pouch, the remaining open edge of the pouch is also heat-sealed. Empty bags are also made to serve as controls. Three samples (i.e., filled and sealed bags) are prepared for the test. The filled bags must be tested within three minutes of preparation unless immediately placed in a sealed container, in which case the filled bags must be tested within thirty minutes of preparation.

The bags are placed between two TEFLON coated fiberglass screens having 3 inch (7.6 cm) openings (available from Taconic Plastics, Inc., having a place of business in Petersburg, N.Y., U.S.A.) and submerged in a pan of the test solution at 23 degrees Celsius, making sure that the screens are held down until the bags are completely wetted. After wetting, the samples remain in the solution for about 20±1 minutes, at which time they are removed from the solution and centrifuged as described in the paragraph below. The samples after being centrifuged and weighed are placed in the solution again for the next measurement. The samples remain in the solution for about 40±1 minutes, at which time they are removed from the solution, centrifuged and weighed to measure their CRC values at 1 hour (20 minutes+40 minutes=60 minutes). The same samples are used for measuring their CRC values at 2 hours and 4 hours (additional soaking time of 1 hour and 2 hours respectively) in the same procedure. Other soaking time intervals may be used for fibers that are expected to exhibit faster absorption rates. For multiple tests, the pan should be emptied and refilled with fresh test solution after 24 bags have been saturated in the pan.

The wet bags taken from the testing solution at different measuring times are then placed into the basket of a suitable centrifuge capable of subjecting the samples to a g-force of about 350. One suitable centrifuge is a Heraeus LaboFuge 400 having a water collection basket, a digital rpm gauge, and a machined drainage basket adapted to hold and drain the bag samples. Where multiple samples are centrifuged, the samples must be placed in opposing positions within the centrifuge to balance the basket when spinning. The bags (including the wet, empty bags) are centrifuged at about 1,600 rpm (e.g., to achieve a target g-force of about 350), for 3 minutes. The bags are removed and weighed, with the empty bags (controls) being weighed first, followed by the bags containing the samples. The amount of solution retained by the sample, taking into account the solution retained by the bag itself, is the centrifuge retention capacity (CRC) of the sample, expressed as grams of fluid per gram of sample. More particularly, the retention capacity is determined as:

$$CRC = \frac{\begin{matrix}s ample \text{/} bag wt after centrifuge - \\ empty bag wt after centrifuge - dry sample wt\end{matrix}}{dry sample wt}$$

where “wt” in the above equation stands for weight.

The three samples are tested and the results are averaged to determine the centrifuge retention capacity (CRC). The samples are tested at 23±1° C. and at 50±2% relative humidity. Unless otherwise stated, all CRC values refer to the value after 4 hours of soaking time.

### Capacity Absorption Rate Test

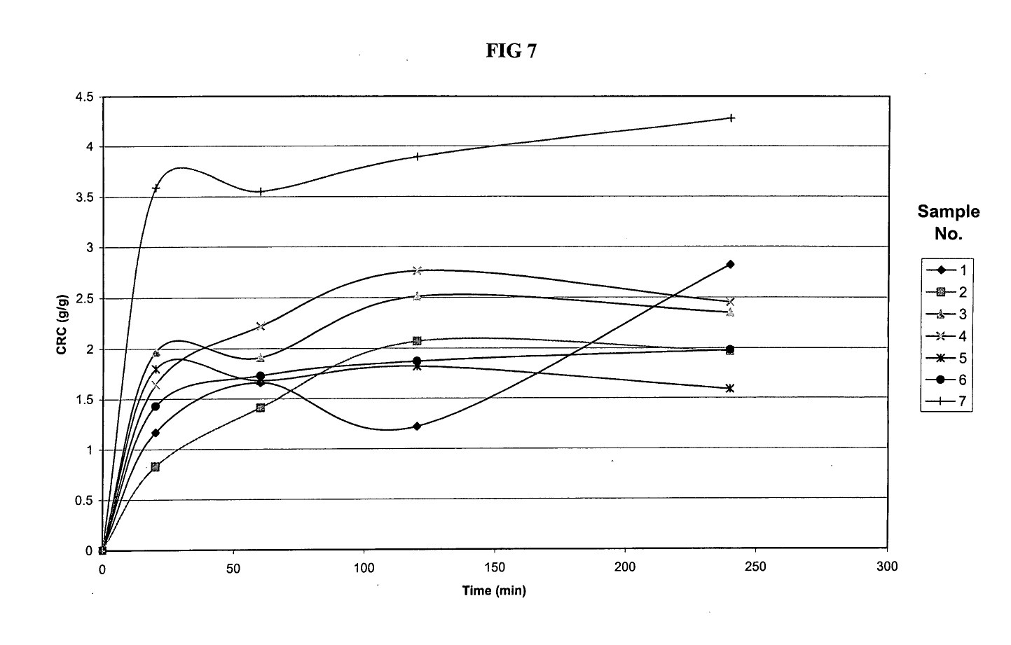

The absorption rate of the material is determined in the following manner. The CRC value after 4 hours of soaking time is multiplied by 60%. A graph of the CRC value as a function of time is then used to determine the time required for the material to swell to 60% of its 4 hour value (e.g., FIG. 7). This is done by plotting the data for 0, 20 minutes, 1 hour, 2 hours, and 4, or other appropriate time intervals as discussed in the Centrifuge Retention Capacity Test above, hours using MICROSOFT OFFICE EXCEL 2003 spreadsheet program, using the standard graphing function with smoothed lines connecting data points, and interpolating the data as necessary, as would be obvious to one skilled in the art. The absorbent rate is calculated by taking the reciprocal of the time (in seconds) required to swell to 60% of its 4 hour value.

### Definitions

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to devices which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, household/industrial absorbent articles, and sports/construction absorbent articles.

The term “coform” is intended to describe a blend of meltblown fibers and cellulose fibers that is formed by air-forming a meltblown polymer material while simultaneously blowing air-suspended cellulose fibers into the stream of meltblown fibers. The coform material may also include other materials, such as superabsorbent materials. The meltblown fibers containing wood fibers and/or other materials are collected on a forming surface, such as provided by a foraminous belt. The forming surface may include a gas-pervious material, such as spunbonded fabric material, that has been placed onto the forming surface.

The terms “elastic,” “elastomeric,” “elastically” and “elastically extensible” are used interchangeably to refer to a material or composite that generally exhibits properties which approximate the properties of natural rubber. The elastomeric material is generally capable of being extended or otherwise deformed, and then recovering a significant portion of its shape after the extension or deforming force is removed.

The term “extensible” refers to a material that is generally capable of being extended or otherwise deformed, but which does not recover a significant portion of its shape after the extension or deforming force is removed.

The terms “fluid impermeable,” “liquid impermeable,” “fluid impervious” and “liquid impervious” mean that fluid such as water or bodily fluids will not pass substantially through the layer or laminate under ordinary use conditions in a direction generally perpendicular to the plane of the layer or laminate at the point of fluid contact.

The term “health/medical absorbent articles” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “household/industrial absorbent articles” includes construction and packaging supplies, products for cleaning and disinfecting, wipes, covers, filters, towels, disposable cutting sheets, bath tissue, facial tissue, nonwoven roll goods, home-comfort products including pillows, pads, mats, cushions, masks and body care products such as products used to cleanse or treat the skin, laboratory coats, coveralls, trash bags, stain removers, topical compositions, pet care absorbent liners, laundry soil/ink absorbers, detergent agglomerators, lipophilic fluid separators, and the like.

The terms “hydrophilic” and “wettable” are used interchangeably to refer to a material having a contact angle of water in air of less than 90 degrees. The term “hydrophobic” refers to a material having a contact angle of water in air of at least 90 degrees. For the purposes of this application, contact angle measurements are determined as set forth in Robert J. Good and Robert J. Stromberg, Ed., in “Surface and Colloid Science—Experimental Methods,” Vol. II, (Plenum Press, 1979), which is hereby incorporated by reference in a manner that is consistent herewith.

The term “layer” when used in the singular can have the dual meaning of a single element or a plurality of elements.

The term “MD” or “machine direction” refers to the orientation of the absorbent web that is parallel to the running direction of the forming fabric and generally within the plane formed by the forming surface. The term “CD” or “cross-machine direction” refers to the direction perpendicular to the MD and generally within the plane formed by the forming surface. Both MD and CD generally define a plane that is parallel to the forming surface. The term “ZD” or “Z-direction” refers to the orientation that is perpendicular to the plane formed by the MD and CD.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. In the particular case of a coform process, the meltblown fiber stream intersects with one or more material streams that are introduced from a different direction. Thereafter, the meltblown fibers and other materials are carried by the high velocity gas stream and are deposited on a collecting surface. The distribution and orientation of the meltblown fibers within the formed web is dependent on the geometry and process conditions. Under certain process and equipment conditions, the resulting fibers can be substantially “continuous,” defined as having few separations, broken fibers or tapered ends when multiple fields of view are examined through a microscope at 10× or 20× magnification. When “continuous” melt blown fibers are produced, the sides of individual fibers will generally be parallel with minimal variation in fiber diameter within an individual fiber length. In contrast, under other conditions, the fibers can be overdrawn and strands can be broken and form a series of irregular, discrete fiber lengths and numerous broken ends. Retraction of the once attenuated broken fiber will often result in large clumps of polymer.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polyolefin” as used herein generally includes, but is not limited to, materials such as polyethylene, polypropylene, polyisobutylene, polystyrene, ethylene vinyl acetate copolymer and the like, the homopolymers, copolymers, terpolymers, etc., thereof, and blends and modifications thereof. The term “polyolefin” shall include all possible structures thereof, which includes, but is not limited to, isotatic, synodiotactic and random symmetries. Copolymers include random and block copolymers.

The term “sports/construction absorbent articles” includes headbands, wrist bands and other aids for absorption of perspiration, absorptive windings for grips and handles of sports equipment, and towels or absorbent wipes for cleaning and drying off equipment during use.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stretchable” refers to materials which may be extensible or which may be elastically extensible.

The terms “superabsorbent” refers to water-swellable, water-insoluble organic or inorganic materials capable, under the most favorable conditions, of absorbing at least about 10 times their weight, or at least about 15 times their weight, or at least about 25 times their weight in an aqueous solution containing 0.9 weight percent sodium chloride. In contrast, “absorbent materials” are capable, under the most favorable conditions, of absorbing at least 2 times their weight, or at least about 5 times their weight of an aqueous solution containing 0.9 weight percent sodium chloride.

The terms “surface treated” and “surface crosslinked” refer to a fiber of the present invention having its molecular chains present in the vicinity of the fiber surface crosslinked by a compound applied to the surface of the fiber. The term “surface crosslinking” means that the functional crosslinks are in the vicinity of the surface of the fiber. As used herein, “surface” describes the outer-facing boundaries of the fiber.

The term “target zone” refers to an area of an absorbent core where it is particularly desirable for the majority of a fluid insult, such as urine, menses, or bowel movement, to initially contact. In particular, for an absorbent core with one or more fluid insult points in use, the insult target zone refers to the area of the absorbent core extending a distance equal to 15% of the total length of the composite from each insult point in both directions.

The term “thermoplastic” describes a material that softens when exposed to heat and which substantially returns to a non-softened condition when cooled to room temperature.

These terms may be defined with additional language in the remaining portions of the specification.

### DETAILED DESCRIPTION

Provided is an absorbent fiber which has a relatively low Centrifuge Retention Capacity (CRC) compared to conventional superabsorbent material, but a higher Centrifuge Retention Capacity and significantly slower absorption rate when compared to untreated fluff fiber. More particularly, the absorbent fiber of the present invention comprises a cellulose fiber that has been modified via chemical modification, wherein the chemical modification can be carboxymethylation, sulfonation, sulfation, phosphonation or the like. The absorbent fiber is substantially water-insoluble and water-swellable. The absorbent fiber may be from a regenerated or a non-regenerated cellulose. Additionally, the absorbent fiber may have the appearance of a conventional cellulose fiber or of a regenerated cellulose fiber. The absorbent fiber exhibits a Centrifuge Retention Capacity after at least 4 hours of between 1.4 and 8 grams per gram (g/g) and has an aborption rate between 0.0001 and 0.01 sec⁻¹. Also provided is an absorbent article comprising the absorbent fiber of the present invention.

Desirably, the absorbent fiber is used in conjunction with superabsorbent material to help absorb extra fluid when the superabsorbent material, such as in a target area of an absorbent article, becomes oversaturated. For example, superabsorbent material typically exhibits a CRC of about 30 grams/gram and exhibits an absorption rate of about 0.01 sec⁻¹. For another example, wood pulp fluff fiber typically exhibits a CRC of less than about 1 gram/gram and exhibits an absorption rate of about 0.2 sec⁻¹. As a result, the superabsorbent material quickly absorbs large quantities of fluid, but then locks it up, preventing it from being distributed to more remote regions from the point of insult. As a result, the superabsorbent material can become oversaturated over time, leading to less absorbent efficiency of an absorbent article, as well as eventual leakage, such as during an insult.

In comparison, the absorbent fiber of the present invention exhibits a CRC of between 1.4 and 10 g/g, such as between 1.4 and 8 g/g, or between 2 and 8 g/g, or between 3 and 8 g/g with an absorption rate of between 0.0001 and 0.01 sec⁻¹. The application of such slower and lower capacity absorbent fiber not only assists in absorbing and retaining fluid, such as from oversaturated superabsorbent material, but also promotes fluid distribution within the article to enhance overall utilization efficiency. The absorbent fiber can additionally retain the fluid both inside and outside of the target zone, which helps prevent leakage of the article. Therefore, the use of the absorbent fiber of the present invention in conjunction with superabsorbent material can result in an absorbent article which provides desirable overall absorbent intake and capacity, as well as improved fluid distribution (as compared to a conventional absorbent article which does not include the absorbent fiber of the present invention). This, in turn, can result in greater performance of the article as well as greater comfort and confidence among the user

In desired aspects of the invention, cellulose fiber (e.g., wood pulp fiber) can be utilized as a base material. Suitable cellulosic fiber to be utilized as base material may include, but is not limited to, chemical wood pulp such as sulfite and sulfate (sometimes called Kraft) pulp, as well as mechanical pulp such as ground wood, thermomechanical pulp and chemithermomechanical pulp. More particularly, the pulp fiber may include cotton, other typical wood pulps, debonded chemical wood pulp, and combinations thereof. Pulp derived from both deciduous and coniferous trees can be used. Additionally, the cellulosic fiber may include such hydrophilic material as natural plant fiber, milkweed floss, cotton fiber, microcrystalline cellulose, microfibrillated cellulose, or any of these materials in combination with wood pulp fiber. Regenerated cellulosic fiber is also suitable for this invention. More particularly, regenerated cellulose fiber include rayon, sulfonated cellulose, carboxymethyl cellulose, and cellulose acetate.

Suitable cellulosic fluff fiber can include, for example, NB480 (available from Weyerhaeuser Co.); NB416, a bleached southern softwood Kraft pulp (available from Weyerhaeuser Co.); CR 54, a bleached southern softwood Kraft pulp (available from Bowater Inc., a business having offices located in Greenville, S.C. U.S.A.).; SULPHATATE HJ, a chemically modified hardwood pulp (available from Rayonier Inc., a business having offices located in Jesup, Ga. U.S.A.); NF 405, a chemically treated bleached southern softwood Kraft pulp (available from Weyerhaeuser Co.); and CR 1654, a mixed bleached southern softwood and hardwood Kraft pulp (available from Bowater Inc. a business having offices located in Greenville, S.C. U.S.A.)

Cellulosic fibers having a wide range of degree of polymerization are suitable for forming the fiber of the invention. In one aspect, the cellulosic fiber has a relatively high degree of polymerization, greater than about 1000, and in another aspect, about 1500 to about 3000. Higher DP cellulosic fibers can be a desirable starting material for the invention because they generally yield modified cellulose fiber with higher absorbent capacity.

In some aspects, the fibers have an average length greater than about 1.0 mm. Consequently, the fibers are suitably prepared from fibers having lengths greater than about 1.0 mm. Fibers having lengths suitable for preparing the fibers include southern pine, northern softwood, and eucalyptus fibers, the average length of which is about 2.8 mm, about 2.0 mm, and about 1.0 mm, respectively.

Cellulose fiber in its conventional (i.e., chemically unmodified) state exhibits a CRC of about 1 g/g or less in 0.9 wt % NaCl saline, which is not desirable from an absorbency standpoint, since only a minimum amount of fluid can be absorbed by the fiber. Thus, the low retention capacity can result in leakage of the article, as well as reduced confidence in the user. The cellulose fiber is then modified via chemical modification to introduce electrolyte functional groups onto the cellulose macromolecules to enhance absorbency. Suitable functional group modifications include, but are not limited to, carboxymethylation, sulfonation, sulfation, phosphonation, and the like, which are capable of enhancing absorbency of the modified wood pulp fibers.

Suitable functional group modification agents for carboxyalkylation include monochloroacetic acid and its salts, and 3-chloropropionic acid and its salts. The carboxyalkyl celluloses useful in preparing the fibers of the invention include carboxymethyl celluloses, carboxyethyl celluloses and carboxymethyl ethyl celluloses.

In one aspect, the present invention provides substantially water-insoluble, water-swellable, chemically modified cellulose fibers. In some aspects, the fibers are non-regenerated. In other aspects, the fibers have a surface having the appearance of the surface of a cellulose fiber. The modified cellulose fibers of the invention are fibers having absorbent properties. The absorbent fibers are water-swellable, water-insoluble fibers that substantially retain a fibrous structure in their expanded, water-swelled state.

The absorbent fibers of the invention are cellulosic fibers that have been modified by carboxymethylation, sulfonation, sulfation, phosphonation, and the like. In some aspects, the absorbent fibers also can be crosslinking, depending on the degree of substitution (DS) and modified functional groups. For example, when a carboxymethyl cellulose fiber has a DS of less than about 0.5, its molecules tend to form crystalline domains within its supermolecular structure which act as crosslinking points. Therefore, no additional chemical crosslinking agent is needed for this low DS fiber to achieve water insolubility. For another example, when carboxymethyl cellulose fiber has a DS of greater than about 0.5, it is water soluble in its sodium salt form. However, this carboxymethyl cellulose fiber becomes water insoluble if it is completely acidified to free acid form. It is believed that the high DS carboxymethyl cellulose fiber in free acid form exhibits low capacity due to lack of electrolyte functional groups and forms crystalline domains which crosslink the fiber through physical means.

Water swellability is imparted to the fibers through the chemical modification and, in some aspects, crosslinking can help render the fibers substantially insoluble in water. For example, when carboxymethylation is selected as the chemical modification, the fibers have a degree of carboxyl group substitution effective to provide advantageous water swellability. The fibers are crosslinked to an extent sufficient to render the fiber water-insoluble. The fibers have a liquid absorption capacity that is increased compared to unmodified fluff pulp fibers.

The fibers are substantially insoluble in water. As used herein, fibers are considered to be water soluble when they substantially dissolve in excess water to form a solution, losing their fiber form and becoming essentially evenly dispersed throughout the water solution. For example, sufficiently carboxyalkylated cellulosic fibers that are free from a substantial degree of crosslinking and are in their sodium salt form will be water soluble, whereas the fibers of the invention, carboxyalkylated and crosslinked fibers, are substantially water insoluble.

The absorbent fibers of the invention are substantially water-insoluble, water-swellable fibers. As used herein, the term “substantially water-insoluble, water-swellable” refers to fibers that, when exposed to an excess of an aqueous medium (e.g., bodily fluids such as urine or blood, water, synthetic urine, or 0.9 weight percent solution of sodium chloride in water), swell to an equilibrium volume, but do not dissolve into solution.

In some aspects, the water-swellable, water-insoluble fibers of the invention have a surface having the appearance of the surface of an unmodified cellulose fiber, such as when a non-regenerated base fiber is used. Like native fibers, these absorbent fibers can have a surface that includes striations, pits, and pores. Thus, when non-regenerated fibers are used as a base material, the resulting absorbent fibers of the invention retain the surface structure of base fibers because the absorbent fibers of the invention are prepared by methods that do not include dissolving the fibers into solution and then regenerating those fibers from the solution. In contrast, absorbent fibers that are prepared by regeneration from solution substantially lack typical fiber structures present in native fibers. Regenerated fibers lack, among other structural features, surface structure (e.g., striations, pits, and pores).

As used herein, the term “regenerated fiber” refers to a fiber that has been prepared by regeneration (i.e., return to a solid form) from a solution that includes dissolved fiber. The term “non-regenerated” refers to a fiber that has not been dissolved into solution and then regenerated. As noted above, whereas the non-regenerated fibers of the invention substantially retain the surface structure of the cellulose fibers from which they are made, regenerated fibers do not. Both non-regenerated and regenerated fibers are suitable for this invention. However, if a non-regenerated fiber is used for the modification, the resultant absorbent fiber will have the appearance of the surface of an unmodified cellulose fiber, such as native wood pulp fluff fiber surface appearance.

The fibers of the invention are substantially insoluble in water while being capable of absorbing water. The fibers of the invention can be rendered water insoluble by virtue of a plurality of intra-fiber crosslinks.

In some aspects, the fibers of the invention may be intra-fiber crosslinked with a metal crosslink. The metal crosslink arises as a consequence of an associative interaction (e.g., bonding) between functional groups on the fiber's carboxymethyl cellulose polymers (e.g., carboxy, or carboxylate) and a multi-valent metal species. Suitable multi-valent metal species include metal ions having a valency of two or greater and that are capable of forming an associative interaction with a carboxymethyl cellulose polymer (e.g., reactive toward associative interaction with the polymer's carboxy, or carboxylate). For example, when carboxymethylation is selected as the chemical modification, the carboxymethyl cellulose polymers are crosslinked when the multi-valent metal species forms an associative interaction with functional groups on the carboxymethyl cellulose polymer. A crosslink may be formed within a carboxymethyl cellulose polymer or may be formed between two or more carboxymethyl cellulose polymers within a fiber. The extent of crosslinking affects the water solubility of the fibers and the ability of the fiber to swell on contact with an aqueous liquid (i.e., the greater the crosslinking, the greater the insolubility). In some aspects, covalent crosslinks derived from reaction of an organic compound having at least two functional groups capable of reacting with at least one functional group of a cellulose polymer (e.g., a diether crosslink derived from crosslinking cellulose with a dihalide such as 1,3-dichloro-2-propanol, or a diester crosslink derived from crosslinking cellulose with citric acid) may be present.

Suitable crosslinking agents useful in making ether crosslinks include dihalide crosslinking agents, such as 1,3-dichloro-2-propanol; diepoxide crosslinking agents, such as vinylcyclohexene dioxide, butadiene dioxide, and diglycidyl ethers (e.g., glycerol diglycidal, 1,4-butanediol diglycidal, and poly(ethylene glycol diglycidal)); sulfone compounds such as divinyl sulfone; bis(2-hydroxyethyl)sulfone, bis(2-chloroethyl)sulfone, and disodium tris(β-sulfatoethyl)sulfonium inner salt; and diisocyanates. Other suitable crosslinking agents include urea-based formaldehyde addition products (e.g., N-methylol compounds), polycarboxylic acids and polyamines.

Suitable urea-based crosslinking agents include methylolated ureas, methylolated cyclic ureas, methylolated lower alkyl substituted cyclic ureas, methylolated dihydroxy cyclic ureas, dihydroxy cyclic ureas, and lower alkyl substituted cyclic ureas. Specific preferred urea-based crosslinking agents include dimethylol urea (DMU, bis[N-hydroxymethyl]urea), dimethylolethylene urea (DMEU, 1,3-dihydroxymethyl-2-imidazolidinone), dimethyloldihydroxyethylene urea (DMDHEU, 1,3-dihydroxymethyl-4,5-dihydroxy-2-imidazolidinone), dimethylolpropylene urea (DMPU), dimethylolhydantoin (DMH), dimethyldihydroxy urea (DMDHU), dihydroxyethylene urea (DHEU, 4,5-dihydroxy-2-imidazolidinone), and dimethyldihydroxyethylene urea (DMeDHEU, 4,5-dihydroxy-1,3-dimethyl-2-imidazolidinone).

Polycarboxylic acid crosslinking agents include the use of C₂-C₉ polycarboxylic acids that contain at least two carboxyl groups (e.g., citric acid and oxydisuccinic acid) as crosslinking agents. Suitable polycarboxylic acid crosslinking agents include citric acid, tartaric acid, malic acid, succinic acid, glutaric acid, citraconic acid, itaconic acid, tartrate monosuccinic acid, maleic acid, 1,2,3-propane tricarboxylic acid, 1,2,3,4-butanetetracarboxylic acid, all-cis-cyclopentane tetracarboxylic acid, tetrahydrofuran tetracarboxylic acid, 1,2,4,5-benzenetetracarboxylic acid, and benzenehexacarboxylic acid. Other polycarboxylic acids crosslinking agents include polymeric polycarboxylic acids such as poly(acrylic acid), poly(methacrylic acid), poly(maleic acid), poly(methylvinylether-co-maleate) copolymer, poly(methylvinylether-co-itaconate) copolymer, copolymers of acrylic acid, and copolymers of maleic acid. The use of polymeric polycarboxylic acid crosslinking agents such as polyacrylic acid polymers, polymaleic acid polymers, copolymers of acrylic acid, and copolymers of maleic acid is described in U.S. Pat. No. 5,998,511, which is incorporated herein by reference in a manner that is consistent herewith.

Suitable crosslinking agents also include crosslinking agents that are reactive toward carboxylic acid groups. Representative organic crosslinking agents include diols and polyols, diamines and polyamines, diepoxides and polyepoxides, polyoxazoline functionalized polymers, and aminols having one or more amino groups and one or more hydroxy groups. In some aspects, mixtures and/or blends of crosslinking agents can also be used.

The crosslinking agent can include a catalyst to accelerate the bonding reaction between the crosslinking agent and cellulosic fiber. Suitable catalysts include acidic salts, such as ammonium chloride, ammonium sulfate, aluminum chloride, magnesium chloride, and alkali metal salts of phosphorous-containing acids.

The amount of optional crosslinking agent applied to the cellulosic fiber will depend on the particular crosslinking agent and is suitably in the range of from about 0.01 to about 10.0% by weight based on the total weight of cellulosic fiber. In one aspect, the amount of crosslinking agent applied to the fibers can be in the range from about 0.5 to about 8.0% by weight based on the total weight of fibers. The amount of crosslinking agent also depends on the degree of substitution (DS) of those chemical modification functional groups, the functional groups themselves (i.e., carboxylate, sulphonate, sulfate or phosphate), or whether the absorbent fiber is in salt or acid forms. For example, when the DS is below 0.5 for a carboxyalkyl cellulose fiber both in salt and acid form, the modified cellulose is usually water-insoluble due to presence of native crystalline domains. Therefore, no crosslinker is required. In contrast, when the DS is above 0.5, the modified cellulose, if it is in sodium salt form, gradually becomes water-soluble, and a crosslinker may be needed.

The fibers of the invention can be characterized as having an average degree of carboxyl group substitution of from about 0.1 to about 0.7. In one aspect, the fibers have an average degree of carboxyl group substitution of from about 0.2 to about 0.6. In another aspect, the fibers have an average degree of carboxyl group substitution of from about 0.2 to about 0.5. As used herein, the “average degree of carboxyl group substitution” refers to the average number of moles of carboxyl groups per mole of glucose unit in the fiber. It will be appreciated that the fibers of the present invention include a distribution of carboxyl functional groups having an average degree of carboxyl substitution, as noted above.

The need for a crosslinking agent is also dependent upon other factors, such as the degree of acidification and the pKa of the chemical modification functional groups. For example, when the degree of acidification increases, the need for a crosslinking agent decreases. Also, when the pKa value increases (i.e., moving toward more weakly acidic), the need to use a crosslinking agent decreases.

In one aspect, the crosslinking agent can be applied to the cellulosic fibers as an aqueous alcoholic solution. Water is present in the solution in an amount sufficient to swell the fiber to an extent to allow for crosslinking within the fiber's cell wall. However, the solution does not include enough water to dissolve or significantly swell the fiber. Suitable alcohols include those alcohols in which the crosslinking agent is soluble and the fiber to be crosslinked (i.e., unmodified or modified cellulosic fiber) is not. Representative alcohols include alcohols that include from 1 to 5 carbon atoms, for example, methanol, ethanol, n-propanol, isopropanol, n-butanol, isobutanol, s-butanol, and pentanols.

It will be appreciated that due to their fibers' structure, the fibers of the invention can have a distribution of functional group and/or crosslinking groups along the fiber's length and through the fiber's cell wall. Generally, there can be greater functional group modification and/or crosslinking on or near the fiber surface than at or near the fiber core. Surface crosslinking may be advantageous to improve fiber dryness and provide a better balance of total absorbent capacity and surface dryness. Fiber swelling and soak time can also affect the functional group modification and crosslinking gradients. Such gradients may be due to the fiber structure and can be adjusted and optimized through control of functional group modification and/or crosslinking reaction conditions.

As referenced above, the absorbent fiber of the present invention can have an absorption rate of between 0.0001 and 0.01 sec⁻¹. This absorption rate can be influenced by a combination of the degree of acidification and pKa of the modified functional groups. For example, a higher degree of acidification can result in a slower absorption rate of the modified absorbent fiber. Thus, a 100% acidified absorbent fiber will have a slower absorption rate compared to a 50% acidified fiber. Similarly, a higher value of pKa can result in a slower absorption rate of the absorbent fiber. For example, sulfonated cellulose will have a faster absorption rate than carboxymethylated cellulose at the same degree of acidification and substitution since sulfonated functional groups are more acidic or have a higher degree of ionization than carboxymethyl functional groups.

In some aspects, a chemical reagent can optionally be used together with the modified absorbent fiber to alter both absorbency and absorption rate. When the degree of substitution and acidification are controlled only on the absorbent fiber of the present invention, it is easy to achieve either high CRC but fast absorption rate, or low CRC but slow absorption rate. In this instance, it is difficult to achieve (1) higher capacity but slow absorption rate, or (2) low capacity but fast absorption rate. Incorporation of a second material can provide more opportunities than the single material. For example, when a high DS, but fully acidified, modified absorbent fiber having a low initial capacity and a slow absorption rate is incorporated with a basic reagent, the reagent can gradually neutralize those acidified functional groups back to a neutralized version and provide high absorbency after a slow process of ion exchanging and neutralization. This composition can result in a high capacity and slow absorption rate. Suitable reagents are well known to those skilled in the art, and can include, but are not limited to, any basic material, such as sodium bicarbonate, sodium carbonate, aluminum oxide, barium hydroxide, polyamine, polyimine, polyquarternay ammonium, chitosan, and the like, including those listed in U.S. Pat. No. 6,951,895B1, which is hereby incorporated by reference in a manner that is consistent herewith.

When a chemical reagent is used, it can be incorporated onto other components of an absorbent product (encapsulation) to achieve several benefits. For example, the amount of material for production handling can be reduced. Also, it can provide an extra control releasing mechanism for absorption rate adjustment. For example, the chemical reagent can be encapsulated into the internal structure of regular wood pulp fluff so that the absorbent fiber of this invention can be mixed with the encapsulated fluff fiber to achieve desired capacity and rate. For another example, the chemical reagent can be coated onto diaper components prior to diaper production, such as surge or fluff fiber materials.

As mentioned above, the absorbent fiber of the present invention can be utilized in an absorbent article. An absorbent article of the present invention can have a topsheet, a backsheet, and an absorbent core disposed between the topsheet and the backsheet. In one aspect, at least one component of the article, such as the absorbent core, includes the absorbent fiber of the present invention. In some aspects, at least one of the topsheet, backsheet, and absorbent core is stretchable.

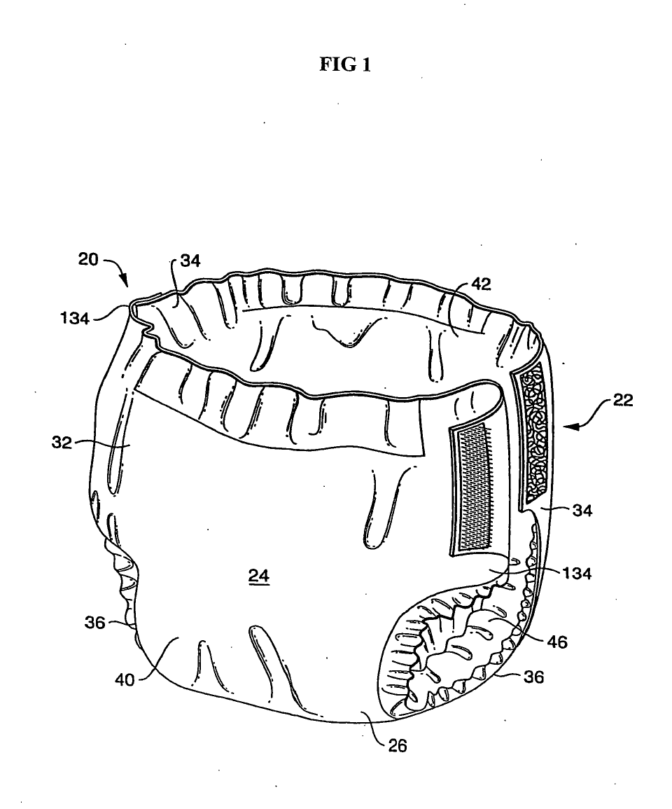

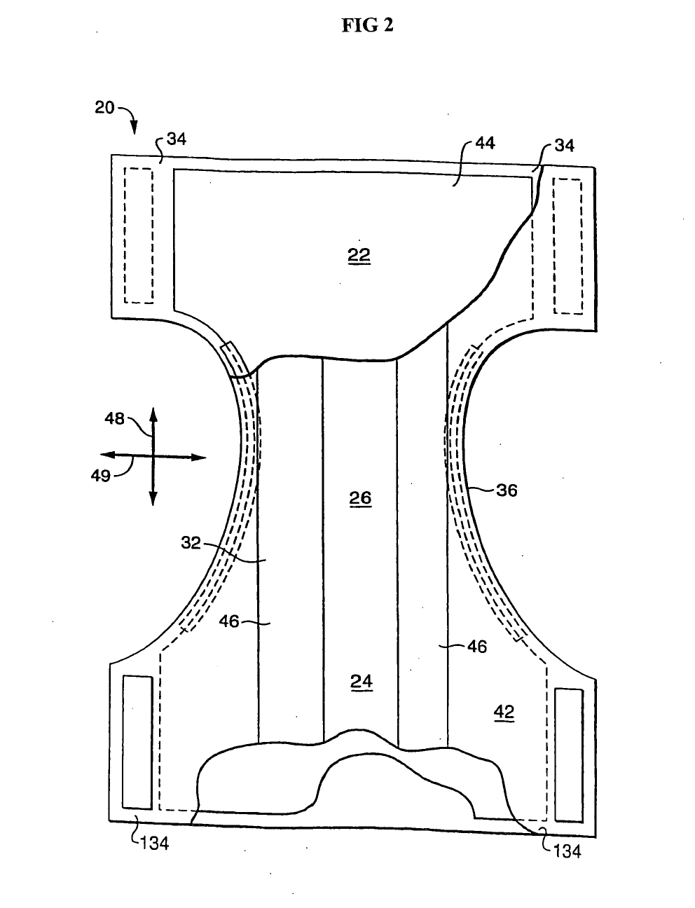

To gain a better understanding of the present invention, attention is directed to FIG. 1 and FIG. 2 for exemplary purposes showing a training pant of the present invention. It is understood that the present invention is suitable for use with various other absorbent articles, including but not limited to other personal care absorbent articles, health/medical absorbent articles, household/industrial absorbent articles, sports/construction absorbent articles, and the like, without departing from the scope of the present invention.

Various materials and methods for constructing training pants are disclosed in PCT Patent Application WO 00/37009 published Jun. 29, 2000 by A. Fletcher et al.; U.S. Pat. Nos. 4,940,464 to Van Gompel et al.; 5,766,389 to Brandon et al., and 6,645,190 to Olson et al., all of which are incorporated herein by reference in a manner that is consistent herewith.

FIG. 1 illustrates a training pant in a partially fastened condition, and FIG. 2 illustrates a training pant in an opened and unfolded state. The training pant defines a longitudinal direction 48 that extends from the front of the training pant when worn to the back of the training pant. Perpendicular to the longitudinal direction 48 is a lateral direction 49.

The pair of training pants defines a front region 22, a back region 24, and a crotch region 26 extending longitudinally between and interconnecting the front and back regions. The pant also defines an inner surface adapted in use (e.g., positioned relative to the other components of the pant) to be disposed toward the wearer, and an outer surface opposite the inner surface. The training pant has a pair of laterally opposite side edges and a pair of longitudinally opposite waist edges.

The illustrated pant 20 may include a chassis 32, a pair of laterally opposite front side panels 34 extending laterally outward at the front region 22 and a pair of laterally opposite back side panels 134 extending laterally outward at the back region 24.

The chassis 32 includes a backsheet 40 and a topsheet 42 that may be joined to the backsheet 40 in a superimposed relation therewith by adhesives, ultrasonic bonds, thermal bonds or other conventional techniques. The chassis 32 may further include an absorbent core 44 such as shown in FIG. 1 disposed between the backsheet 40 and the topsheet 42 for absorbing fluid body exudates exuded by the wearer, and may further include a pair of containment flaps 46 secured to the topsheet 42 or the absorbent core 44 for inhibiting the lateral flow of body exudates.

The backsheet 40, the topsheet 42 and the absorbent core 44 may be made from many different materials known to those skilled in the art. All three layers, for instance, may be extensible and/or elastically extensible. Further, the stretch properties of each layer may vary in order to control the overall stretch properties of the product.

The backsheet 40, for instance, may be breathable and/or may be fluid impermeable. The backsheet 40 may be constructed of a single layer, multiple layers, laminates, spunbond fabrics, films, meltblown fabrics, elastic netting, microporous webs or bonded-carded-webs. The backsheet 40, for instance, can be a single layer of a fluid impermeable material, or alternatively can be a multi-layered laminate structure in which at least one of the layers is fluid impermeable.

The backsheet 40 can be biaxially extensible and optionally biaxially elastic. Elastic non-woven laminate webs that can be used as the backsheet 40 include a non-woven material joined to one or more gatherable non-woven webs or films. Stretch Bonded Laminates (SBL) and Neck Bonded Laminates (NBL) are examples of elastomeric composites.

Examples of suitable nonwoven materials are spunbond-meltblown fabrics, spunbond-meltblown-spunbond fabrics, spunbond fabrics, or laminates of such fabrics with films, or other nonwoven webs. Elastomeric materials may include cast or blown films, meltblown fabrics or spunbond fabrics composed of polyethylene, polypropylene, or polyolefin elastomers, as well as combinations thereof. The elastomeric materials may include PEBAX elastomer (available from AtoFina Chemicals, Inc., a business having offices located in Philadelphia, Pa. U.S.A.), HYTREL elastomeric polyester (available from Invista, a business having offices located in Wichita, Kans. U.S.A.), KRATON elastomer (available from Kraton Polymers, a business having offices located in Houston, Tex., U.S.A.), or strands of LYCRA elastomer (available from Invista), or the like, as well as combinations thereof. The backsheet 40 may include materials that have elastomeric properties through a mechanical process, printing process, heating process or chemical treatment. For example, such materials may be apertured, creped, neck-stretched, heat activated, embossed, and micro-strained, and may be in the form of films, webs, and laminates.

One example of a suitable material for a biaxially stretchable backsheet 40 is a breathable elastic film/nonwoven laminate, such as described in U.S. Pat. No. 5,883,028, to Morman et al., incorporated herein by reference in a manner that is consistent herewith. Examples of materials having two-way stretchability and retractability are disclosed in U.S. Pat. Nos. 5,116,662 to Morman and 5,114,781 to Morman, each of which is incorporated herein by reference in a manner that is consistent herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The topsheet 42 is suitably compliant, soft-feeling and non-irritating to the wearer's skin. The topsheet 42 is also sufficiently liquid permeable to permit liquid body exudates to readily penetrate through its thickness to the absorbent core 44. A suitable topsheet 42 may be manufactured from a wide selection of web materials, such as porous foams, reticulated foams, apertured plastic films, woven and non-woven webs, or a combination of any such materials. For example, the topsheet 42 may include a meltblown web, a spunbonded web, or a bonded-carded-web composed of natural fibers, synthetic fibers or combinations thereof. The topsheet 42 may be composed of a substantially hydrophobic material, and the hydrophobic material may optionally be treated with a surfactant or otherwise processed to impart a desired level of wettability and hydrophilicity.

The topsheet 42 may also be extensible and/or elastomerically extensible. Suitable elastomeric materials for construction of the topsheet 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio U.S.A.), or PEBAX elastomers. The topsheet 42 can also be made from extensible materials such as those described in U.S. Pat. No. 6,552,245 to Roessler et al. which is incorporated herein by reference in a manner that is consistent herewith. The topsheet 42 can also be made from biaxially stretchable materials as described in U.S. Pat. No. 6,641,134 filed to Vukos et al. which is incorporated herein by reference in a manner that is consistent herewith.

The article 20 can optionally further include a surge management layer which may be located adjacent the absorbent core 44 and attached to various components in the article 20 such as the absorbent core 44 or the topsheet 42 by methods known in the art, such as by using an adhesive. In general, a surge management layer helps to quickly acquire and diffuse surges or gushes of liquid that may be rapidly introduced into the absorbent structure of the article. The surge management layer can temporarily store the liquid prior to releasing it into the storage or retention portions of the absorbent core 44. Examples of suitable surge management layers are described in U.S. Pat. Nos. 5,486,166 to Bishop et al.; 5,490,846 to Ellis et al.; and 5,820,973 to Dodge et al., each of which is incorporated herein by reference in a manner that is consistent herewith.

The article 20 can further comprise an absorbent core 44. The absorbent core 44 may have any of a number of shapes. For example, it may have a 2-dimensional or 3-dimensional configuration, and may be rectangular shaped, triangular shaped, oval shaped, race-track shaped, 1-shaped, generally hourglass shaped, T-shaped and the like. It is often suitable for the absorbent core 44 to be narrower in the crotch portion 26 than in the rear 24 or front 22 portion(s). The absorbent core 44 can be attached in an absorbent article, such as to the backsheet 40 and/or the topsheet 42 for example, by bonding means known in the art, such as ultrasonic, pressure, adhesive, aperturing, heat, sewing thread or strand, autogenous or self-adhering, hook-and-loop, or any combination thereof.

In some aspects, the absorbent core 44 can have a significant amount of stretchability. For example, the absorbent core 44 can comprise a matrix of fibers which includes an operative amount of elastomeric polymer fibers. Other methods known in the art can include attaching superabsorbent polymer particles to a stretchable film, utilizing a nonwoven substrate having cuts or slits in its structure, and the like.

The absorbent core 44 can be formed using methods known in the art. While not being limited to the specific method of manufacture, the absorbent core can utilize forming drum systems, for example, see U.S. Pat. No. 4,666,647 entitled Apparatus And Method For Forming A Laid Fibrous Web by K. Enloe et al. which issued May 19, 1987, U.S. Pat. No. 4,761,258 entitled Controlled Formation Of Light. And Heavy Fluff Zones by K. Enloe which issued Aug. 2, 1988, U.S. Pat. No. 6,630,088 entitled Forming Media With Enhanced Air Flow Properties by Venturino et al. which issued Oct. 7, 2003, and U.S. Pat. No. 6,330,735 entitled Apparatus And Process For Forming A Laid Fibrous Web With Enhanced Basis Weight. Capability by Hahn et al. which issued Dec. 18, 2001; the entire disclosures of which are incorporated herein by reference in a manner that is consistent herewith. Examples of techniques which can introduce a selected quantity of optional superabsorbent particles into a forming chamber are described in U.S. Pat. No. 4,927,582 entitled Method And Apparatus For Creating A Graduated Distribution Of Granule Materials In A Fiber Mat by R. E. Bryson which issued May 22, 1990 and U.S. Pat. No. 6,416,697 entitled Method For Obtaining A Dual Strata Distribution Of Superabsorbent In A Fibrous Matrix by Venturino et al. which issued Jul. 9, 2002; the entire disclosures of which are incorporated herein by reference in a manner that is consistent herewith.

In some desirable aspects, the absorbent core includes the absorbent fiber of the present invention and superabsorbent material only to provide improved benefits. In other aspects, the absorbent core may further comprise unmodified cellulose fiber and/or synthetic fiber, such as meltblown fiber, for example. Thus, in some aspects, a meltblown process can be utilized, such as to form the absorbent core in a coform line. Exemplary meltblown processes are described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. Nos. 3,849,241 and 5,350,624, all of which are incorporated herein by reference in a manner that is consistent herewith.

To form “coform” materials, additional components are mixed with the meltblown fibers as the fibers are deposited onto a forming surface. For example, the absorbent fibers of the present invention and superabsorbent material may be injected into the meltblown fiber stream so as to be entrapped and/or bonded to the meltblown fibers. Exemplary coform processes are described in U.S. Pat. Nos. 4,100,324 to Anderson et al.; 4,587,154 to Hotchkiss et al.; 4,604,313 to McFarland et al.; 4,655,757 to McFarland et al.; 4,724,114 to McFarland et al.; 4,100,324 to Anderson et al.; and U.K. Patent GB 2,151,272 to Minto et al., each of which is incorporated herein by reference in a manner that is consistent herewith. Absorbent, elastomeric meltblown webs containing high amounts of absorbent materials are described in U.S. Pat. No. 6,362,389 to D. J. McDowall, and absorbent, elastomeric meltblown webs containing high amounts of superabsorbent and low superabsorbent shakeout values are described in pending U.S. patent application Ser. No. 10/883,174 to X. Zhang et al., each of which is incorporated herein by reference in a manner that is consistent herewith.

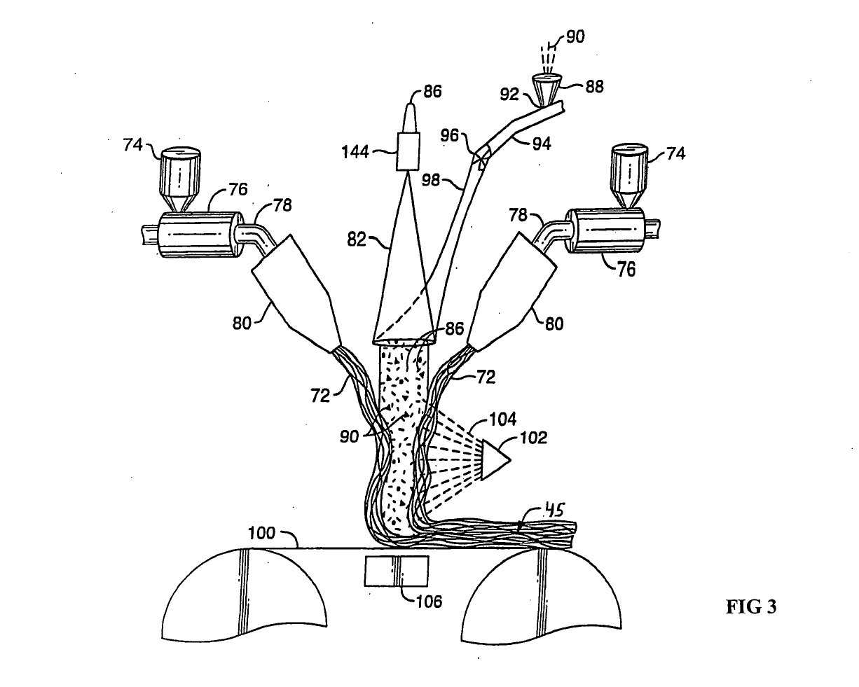

One example of a method of forming an absorbent core 44 for use in the present invention is illustrated in FIG. 3. The dimensions of the apparatus in FIG. 3 are described herein by way of example. Other types of apparatus having different dimensions and/or different structures may also be used to form the absorbent core 44. As shown in FIG. 3, elastomeric material 72 in the form of pellets can be fed through two pellet hoppers 74 into two single screw extruders 76 that each feed a spin pump 78. The elastomeric material 72 may be a multicomponent elastomer blend available under the trade designation VISTMAXX 2370 from ExxonMobil Chemical Company (a business having offices located in Houston, Tex. U.S.A.), as well as others mentioned herein. Each spin pump 78 feeds the elastomeric material 72 to a separate meltblown die 80. Each meltblown die 80 may have 30 holes per inch (hpi). The die angle may be adjusted anywhere between 0 and 70 degrees from horizontal, and is suitably set at about 45 degrees. The forming height may be at a maximum of about 16 inches, but this restriction may differ with different equipment.

A chute 82 having a width of about 24 inches wide may be positioned between the meltblown dies 80. The depth, or thickness, of the chute 82 may be adjustable in a range from about 0.5 to about 1.25 inches (1.3 cm to 3.2 cm), or from about 0.75 to about 1.0 inch (1.9 cm to 2.5 cm). A picker 144 connects to the top of the chute 82. The picker 144 is used to fiberize fibers 86, which may include the absorbent fiber of the present invention, and optionally other cellulose fiber.

At an end of the chute 82 opposite the picker 144 is a superabsorbent material feeder 88. The feeder 88 pours superabsorbent material 90 into a hole 92 in a pipe 94 which then feeds into a blower fan 96. Past the blower fan 96 is a length of 4-inch diameter pipe 98 sufficient for developing a fully developed turbulent flow at about 5,000 feet per minute, which allows the superabsorbent material 90 to become distributed. The pipe 98 widens from a 4-inch (10-cm) diameter to the 24-inch by 0.75-inch (61 cm by 1.9 cm) chute 82, at which point the superabsorbent material 90 mixes with the absorbent fibers 86 and the mixture falls straight down and gets mixed on either side at an approximately 45-degree angle with the elastomeric material 72. The mixture of superabsorbent material 90, absorbent fibers of the present invention 86, and elastomeric material 72 falls onto a wire conveyor 100 moving from about 14 to about 35 feet per minute. However, before hitting the wire conveyor 100, a spray boom 102 optionally sprays an aqueous surfactant mixture 104 in a mist through the mixture, thereby rendering the resulting absorbent core 44 wettable. The surfactant mixture 104 may be a 1:3 mixture of GLUCOPON 220 UP (available from Cognis Corporation having a place of business in Cincinnati, Ohio, U.S.A.) and AHCOVEL Base N-62 (available from Uniqema, having a place of business in New Castle, Del., U.S.A.). An under wire vacuum 106 is positioned beneath the conveyor 100 to assist in forming the absorbent core 44.

As referenced above, the absorbent core 44 includes absorbent and superabsorbent material. Accordingly, the absorbent core 44 can comprise a quantity of absorbent fibers of the present invention, superabsorbent material and optionally fluff contained within a matrix of fibers. In some aspects, the total amount of superabsorbent material in the absorbent core 44 can be at least about 10% by weight of the core, such as at least about 30%, or at least about 60% by weight or at least about 90%, or between about 10% and about 98% by weight of the core, or between about 30% to about 90% by weight of the core to provide improved benefits. Optionally, the amount of superabsorbent material can be at least about 95% by weight of the core. In other aspects, the amount of absorbent fiber of the present invention in the absorbent core 44 can be at least about 5% by weight of the core, such as at least about 30%, or at least about 50% by weight of the core, or between about 5% and 90%, such as between about 10% and 70% or between 10% and 50% by weight of the core. In still other aspects, the absorbent core 44 can optionally comprise about 35% or less by weight unmodified fluff, such as about 20% or less, or 10% or less by weight unmodified fluff.

It should be understood that the present invention is not restricted to use with absorbent fibers of the present invention, superabsorbent material and optionally fluff. In some aspects, the absorbent core 44 may additionally include materials such as surfactants, ion exchange resin particles, moisturizers, emollients, perfumes, natural fibers, synthetic fibers, fluid modifiers, odor control additives, and combinations thereof. In addition, the absorbent core 44 can include a foam.

In order to function well, the absorbent core 44 can have certain desired properties to provide improved performance as well as greater comfort and confidence among the user. For instance, the absorbent core 44 can have corresponding configurations of absorbent capacities, densities, basis weights and/or sizes which are selectively constructed and arranged to provide desired combinations of absorbency properties such as liquid intake rate, absorbent capacity, liquid distribution or fit properties such as shape maintenance and aesthetics. Likewise, the components can have desired wet to dry strength ratios, mean flow pore sizes, permeabilities and elongation values.

As mentioned above, the absorbent core 44 can optionally include elastomeric polymer fibers. The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc. under the trade designation KRATON elastomeric resin or from Dexco, a division of ExxonMobil Chemical Company under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from Invista Corporation under the trade name LYCRA polyurethane, and ESTANE available from Noveon, Inc., a business having offices located in Cleveland, Ohio U.S.A.; thermoplastic elastic polyamides, including polyether block amides available from AtoFina Chemicals, Inc. (a business having offices located in Philadelphia, Pa. U.S.A.) under the trade name PEBAX; polyether block amide; thermoplastic elastic polyesters, including those available from E. I. Du Pont de Nemours Co., under the trade name HYTREL, and ARNITEL from DSM Engineering Plastics (a business having offices located in Evansville, Ind., U.S.A.) and single-site or metallocene-catalyzed polyolefins having a density of less than about 0.89 grams/cubic centimeter, available from Dow Chemical Co. (a business having offices located in Freeport, Tex. U.S.A.) under the trade name AFFINITY; and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS and SEPS. In these copolymers the A blocks are polystyrene and the B blocks are a rubbery component. Generally, these triblock copolymers have molecular weights that can vary from the low thousands to hundreds of thousands, and the styrene content can range from 5% to 75% based on the weight of the triblock copolymer. A diblock copolymer is similar to the triblock, but is of an AB structure. Suitable diblocks include styrene-isoprene diblocks, which have a molecular weight of approximately one-half of the triblock molecular weight having the same ratio of A blocks to B blocks.

In desired arrangements, the polymer fibers can include at least one material selected from the group consisting of styrenic block copolymers, elastic polyolefin polymers and co-polymers and EVA/EMA type polymers.

In some particular arrangements, for example, the elastomeric material of the polymer fibers can include various commercial grades of low crystallinity, lower molecular weight metallocene polyolefins, available from ExxonMobil Chemical Company (a company having offices located in Houston, Tex., U.S.A.) under the VISTAMAXX trade designation. Some VISTAMAXX materials are believed to be metallocene propylene ethylene co-polymer. For example, in one aspect the elastomeric polymer can be VISTAMAXX PLTD 2210. In other aspects, the elastomeric polymer can be VISTAMAXX PLTD 1778. In a particular aspect, the elastomeric polymer is VISTAMAXX 2370. Another optional elastomeric polymer is KRATON blend G 2755 from Kraton Inc. The KRATON material is believed to be a blend of styrene ethylene-butylene styrene polymer, ethylene waxes and tackifying resins.

In some aspects, the elastomeric polymer fibers can be produced from a polymer material having a selected melt flow rate (MFR). In a particular aspect, the MFR can be up to a maximum of about 300. Alternatively, the MFR can be up to about 230 or 250. In another aspect, the MFR can be a minimum of not less than about 9, or not less than 20. The MFR can alternatively be not less than about 50 to provide desired performance. The described melt flow rate has the units of grams flow per 10 minutes (g/10 min). The parameter of melt flow rate is well known, and can be determined by conventional techniques, such as by employing test ASTM D 1238 70 “extrusion plastometer” Standard Condition “L” at 230° C. and 2.16 kg applied force.

As referenced above, the elastomeric polymer fibers of the absorbent core 44 can include an amount of a surfactant. The surfactant can be combined with the elastomeric polymer fibers of the absorbent core in any operative manner. Various techniques for combining the surfactant are conventional and well known to persons skilled in the art. For example, the surfactant may be compounded with the elastomeric polymer employed to form a meltblown fiber structure. In a particular feature, the surfactant may be configured to operatively migrate or segregate to the outer surface of the fibers upon the cooling of the fibers. Alternatively, the surfactant may be applied to or otherwise combined with the elastomeric polymer fibers after the fibers have been formed.

The elastomeric polymer fibers can include an operative amount of surfactant, based on the total weight of the fibers and surfactant. In some aspects, the elastomeric polymer fibers can include at least a minimum of about 0.1% by weight surfactant, as determined by water extraction. The amount of surfactant can alternatively be at least about 0.15% by weight, and can optionally be at least about 0.2% by weight to provide desired benefits. In other aspects, the amount of surfactant can be generally not more than a maximum of about 2% by weight, such as not more than about 1% by weight, or not more than about 0.5% by weight to provide improved performance.

If the amount of surfactant is outside the desired ranges, various disadvantages can occur. For example, an excessively low amount of surfactant may not allow fibers, such as hydrophobic meltblown fibers, to wet with the absorbed fluid. In contrast, an excessively high amount of surfactant may allow the surfactant to wash off from the fibers and undesirably interfere with the ability of the absorbent core to transport fluid, or may adversely affect the attachment strength of the absorbent core to the absorbent article. Where the surfactant is compounded or otherwise internally added to the polymer fibers, an excessively high level of surfactant can create conditions that cause poor formation of the polymer fibers and interfiber bonds.

In some configurations, the surfactant can include at least one material selected from the group that includes polyethylene glycol ester condensates and alkyl glycoside surfactants. For example, the surfactant can be a GLUCOPON surfactant, available from Cognis Corporation, which can be composed of 40 wt. % water, and 60 wt. % d-glucose, decyl, octyl ethers and oligomerics.

In other aspects of the invention, the surfactant can be in the form of a sprayed-on surfactant comprising a water/surfactant solution which includes 16 liters of hot water (about 45° C. to 50° C.) mixed with 0.20 kg of GLUCOPON 220 UP surfactant available from Cognis Corporation and 0.36 kg of AHCHOVEL Base N-62 surfactant available from Uniqema. When employing a sprayed-on surfactant, a relatively lower amount of sprayed-on surfactant may be desirable to provide the desired containment of the superabsorbent polymer particles. Excessive amounts of the fluid surfactant may hinder the desired attachment of the superabsorbent polymer particles to the molten, elastomeric meltblown fibers, for example.

An example of an internal surfactant or wetting agent that can be compounded with the elastomeric fiber polymer can include a MAPEG DO 400 PEG (polyethylene glycol) ester, available from BASF (a business having offices located in Freeport, Tex., U.S.A.). Other internal surfactants can include a polyether, a fatty acid ester, a soap or the like, as well as combinations thereof.

As referenced above, the absorbent core 44 can optionally include a desired amount of superabsorbent material (SAM) of the present invention. SAM typically includes polymers of ethylenically unsaturated carboxylic acids or derivatives thereof. These polymers are rendered water insoluble, but water swellable, by crosslinking the polymer with a di- or polyfunctional internal crosslinking agent. These internally crosslinked polymers are at least partially neutralized and contain pendant anionic carboxyl groups on the polymer backbone that enable the polymer to absorb aqueous fluids, such as body fluids. Typically, the SAM is subjected to a post-treatment to crosslink the pendant anionic carboxyl groups on the surface of the material.

The superabsorbent material can be selected from natural, synthetic and modified natural polymers and materials. The superabsorbent material can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces. Processes for preparing synthetic, absorbent gelling polymers are disclosed in U.S. Pat. No. 4,076,663, issued to Masuda et al., and U.S. Pat. No. 4,286,082, issued to Tsubakimoto et al., all of which are incorporated herein by reference in a manner that is consistent herewith. Suitable superabsorbent material is available from various commercial vendors, such as Stockhausen, Inc., BASF Inc. and others.

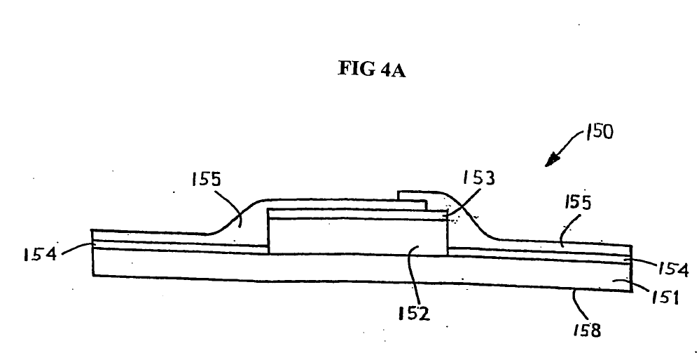

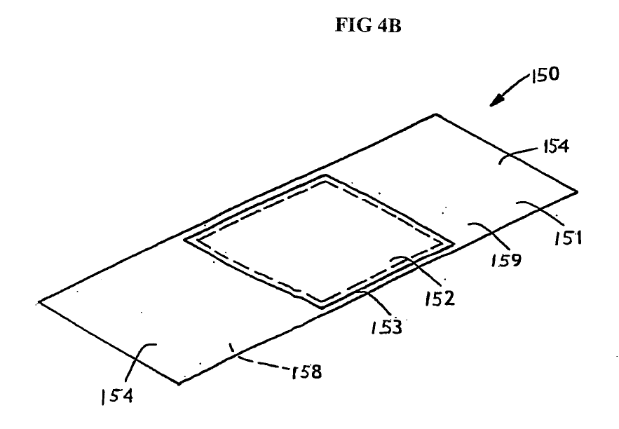

In addition to the absorbent article described above, the present invention may be exemplified as an absorbent bandage. Attention is directed to FIGS. 4A and 4B, which show a possible configuration for a bandage of the present invention. FIG. 4A shows a cross-section view of the absorbent bandage with optional layers described below. FIG. 4B shows a perspective view of the bandage of the present invention with some of the optional or removable layers not being shown. The absorbent bandage 150 has a strip 151 of material having a body-facing side 159 and a second side 158 which is opposite the body-facing side. The strip is essentially a backsheet and is desirably prepared from the same materials described above for the backsheet. In addition, the strip may be an apertured material, such as an apertured film, or material which is otherwise gas permeable, such as a gas permeable film. The strip 151 supports an absorbent core 152 comprising superabsorbent material and absorbent fibers of the present invention which is attached to the body facing side 159 of the strip. In addition, an absorbent protective layer 153 may be applied to the absorbent core 152 and can be coextensive with the strip 151.

The absorbent bandage 150 of the present invention may also have a pressure sensitive adhesive 154 applied to the body-facing side 159 of the strip 151. Any pressure sensitive adhesive may be used, provided that the pressure sensitive adhesive does not irritate the skin of the user. Suitably, the pressure sensitive adhesive is a conventional pressure sensitive adhesive which is currently used on similar conventional bandages. This pressure sensitive adhesive is desirably not placed on the absorbent core 152 or on the absorbent protective layer 153 in the area of the absorbent core 152. If the absorbent protective layer is coextensive with the strip 151, then the adhesive may be applied to areas of the absorbent protective layer 153 where the absorbent core 152 is not located. By having the pressure sensitive adhesive on the strip 151, the bandage is allowed to be secured to the skin of a user in need of the bandage. To protect the pressure sensitive adhesive and the absorbent, a release strip 155 can be placed on the body facing side 159 of the bandage. The release liner may be removably secured to the article attachment adhesive and serves to prevent premature contamination of the adhesive before the absorbent article is secured to, for example, the skin. The release liner may be placed on the body facing side of the bandage in a single piece (not shown) or in multiple pieces, as is shown in FIG. 4A.

In another aspect of the present invention, the absorbent core of the bandage may be placed between a folded strip. If this method is used to form the bandage, the strip is suitably fluid permeable.

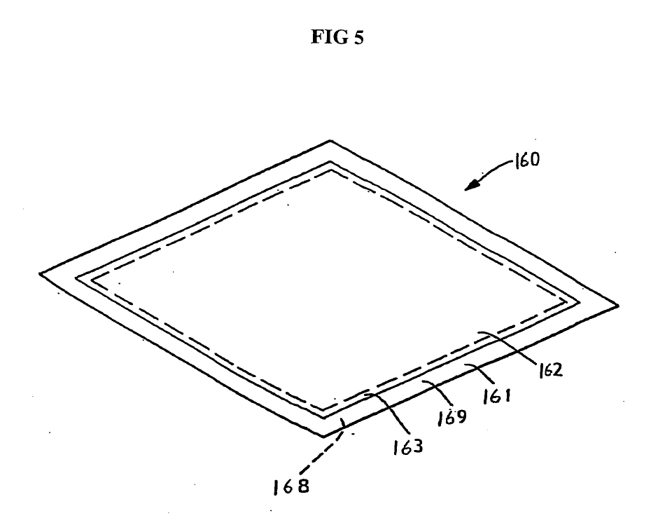

Absorbent furniture and/or bed pads or liners are also included within the present invention. As is shown in FIG. 5, a furniture or bed pad or liner 160 (hereinafter referred to as a “pad”) is shown in perspective. The pad 160 has a fluid impermeable backsheet 161 having a furniture-facing side or surface 168 and an upward facing side or surface 169 which is opposite the furniture-facing side or surface 168. The fluid impermeable backsheet 161 supports the absorbent core 162 which comprises superabsorbent material and absorbent fibers of the present invention, and which is attached to the upward facing side 169 of the fluid impermeable backsheet. In addition, an optional absorbent protective layer 163 may be applied to the absorbent core. The optional substrate layer of the absorbent core can be the fluid impermeable layer 161 or the absorbent protective layer 163 of the pad.

To hold the pad in place, the furniture-facing side 168 of the pad may contain a pressure sensitive adhesive, a high friction coating or other suitable material which will aid in keeping the pad in place during use. The pad of the present invention can be used in a wide variety of applications including placement on chairs, sofas, beds, car seats and the like to absorb any fluid which may come into contact with the pad.

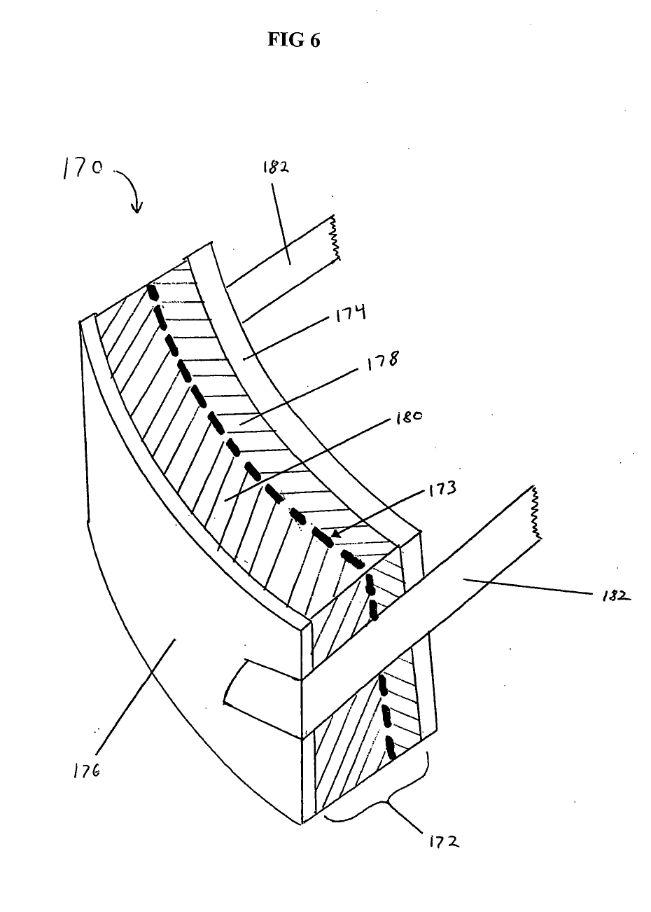

Sports/construction absorbent articles, such as an absorbent headband for absorbing perspiration or drying off equipment are also included within the present invention. As is shown in FIG. 6, a highly absorbent sweatband 170 is shown in perspective. The sweatband 170 has an absorbent core 172 disposed between an optional topsheet 174 and/or an optional fluid impervious backsheet 176. The absorbent core 172 comprises superabsorbent material and absorbent fibers of the present invention, and in some aspects can have a low capacity region 178 and a high capacity region 180, and could include an optional additional region (not shown) if desired. The regions are stratified through polymeric bonding and polymer fiber intermingling, as shown by broken line 173. The sweatband can be useful where dimensional stability is needed to maintain good contact with the skin to intercept perspiration prior to contact with the hands or eyes. The low capacity region 178 can be positioned towards the user's skin and can maintain a comfortable feel to the user. VELCRO or other fastening device 182 can be used to facilitate adjustment or comfort.

The present invention may be better understood with reference to the following examples.

### EXAMPLES

20 grams of dry and fiberized NB-480 wood pulp (available from Weyerhaeuser Company, having a place of business located in Federal Way, Wash., U.S.A.) was mixed vigorously at room temperature with 800 grams of isopropanol in a 2 liter KONTES glass reactor (available from VWR Lab Shop located in Batavia, Ill., U.S.A.) equipped with an agitator and a jacket for temperature control. Dry sodium hydroxide (see table below) was separately dissolved in deionized water. The wood pulp fluff and alcohol suspension was then mixed vigorously while the sodium hydroxide solution was slowly added into the suspension over a 30 minute period. Agitation was continued for another 30 minutes after complete addition of the sodium hydroxide solution.

Dry mono-chloroacetic acid (see Table 1 below) was slowly added into the suspension while the agitation was maintained at the same speed. Addition of the mono-chloroacetic acid was completed within approximately 30 minutes. All of the chemicals used in this fiber modification reaction are commercially available from Aldrich, having a place of business in Milwaukee, Wis., U.S.A. After the addition, a heat bath circulator with programmable digital controller (Model 1197, available from VWR Scientific Product, having a place of business located in Bristol, Conn., U.S.A.) was turned on and the temperature of circulating fluid was set at 60° C. for 3 hours. During this three-hour reaction period, agitation was continued at a speed which ensured uniform mixing of the fluff fiber with the modification chemicals and uniform temperature within the reactor.

After the reaction was completed, the carboxymethyl cellulose fiber produced was washed 3 times with 800 ml of an aqueous methanol solution at a concentration of 70 wt % methanol. A vacuum filtration was used to remove washing liquid after each washing step. Half of the washed fiber was dried at 60° C. for 10 hours while the rest of the fiber was acidified by mixing it in the reactor vigorously at room temperature with 500 ml of 70 wt % methanol and one gram of 37 wt % hydrochloric acid (HCl). The HCl acidified fiber was then washed twice in the same way as previously by 500 ml of 70 wt % methanol to ensure that any residual chemicals were removed. The acidified and washed fiber was then dried at 60° C. for 10 hours.

The above two versions of samples were designated as Na-Type (sodium salt form) and H-Type (acid form), respectively. Table 1 below summarizes the samples produced. Even though no crosslinking agent was used in the modification reaction, all of the carboxymethyl cellulose fibers in the table are generally water-insoluble due to a low degree of substitution and/or acidification. The Na-Type fiber with a high degree of modification tends to have an increased degree of extractables which was reflected by a reduction in centrifuge retention capacity (CRC) as measuring time was increased.

TABLE 1

| | | | | | | | | | | | Absorption |
|---|---|---|---|---|---|---|---|---|---|---|---|
| Sample | Chemicals Used in Fiber Modification (g) | | | | | Fiber | CRC (g/g) | | | | Rate |
| No. | Fluff | Isopopanol | NaOH | H₂O | ClCH₂COOH | Type | 20 min | 1 hr | 2 hr | 4 hr | Sec⁻¹ |
| 1 | 20 | 800 | 4.8 | 14 | 6.6 | Na | 1.17 | 1.66 | 1.22 | 2.82 | 0.0003 |
| 2 | 20 | 800 | 4.8 | 14 | 6.6 | H | 0.83 | 1.41 | 2.07 | 1.97 | 0.0004 |
| 3 | 20 | 800 | 6.4 | 18.4 | 8.8 | Na | 1.97 | 1.91 | 2.51 | 2.35 | 0.0011 |
| 4 | 20 | 800 | 6.4 | 18.4 | 8.8 | H | 1.64 | 2.22 | 2.76 | 2.45 | 0.0008 |
| 5 | 20 | 800 | 9.6 | 27.6 | 13.2 | Na | 1.80 | 1.68 | 1.82 | 1.59 | 0.0014 |
| 6 | 20 | 800 | 9.6 | 27.6 | 13.2 | H | 1.43 | 1.73 | 1.87 | 1.98 | 0.001 |
| 7 | 20 | 800 | 16.0 | 46.0 | 22.0 | H | 3.59 | 3.55 | 3.89 | 4.28 | 0.001 |

Note:
As a reference, commercial superabsorbent material will exhibit a CRC value of around 30 g/g and an absorption rate of about 0.01 sec⁻¹ while commercial unmodified fluff fiber will exhibit a CRC value of less than about 1 g/g and a rate of about 0.2 sec⁻¹.

As can be seen in the above table, samples 1-7 are all examples of the invention, exhibiting a CRC after 4 hours of swelling between 1.4 and 8 g/g and an absorption rate of between 0.0001 and 0.01 sec⁻¹. FIG. 7 is a graph of the CRC value as a function of time for each of the samples.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. An absorbent fiber comprising a cellulose fiber that has been modified via chemical modification;
wherein the chemical modification is selected from carboxymethylation, sulfonation, sulfation or phosphonation;
wherein the absorbent fiber is substantially water-insoluble and water-swellable; and
wherein the absorbent fiber exhibits a Centrifuge Retention Capacity after four hours of between 1.4 and 8 grams per gram and an absorption rate between 0.0001 and 0.01 sec⁻¹.

2. The absorbent fiber of claim 1 further comprising a crosslinking agent.

3. The absorbent fiber of claim 1 having a degree of substitution of less than 0.5.

4. The absorbent fiber of claim 1 having a degree of substitution of greater than 0.5.

5. The absorbent fiber of claim 1 having an average degree of carboxyl group substitution of from about 0.1 to about 0.7.

6. The absorbent fiber of claim 1 further comprising a reagent.

7. The absorbent fiber of claim 1 wherein the cellulose fiber is non-regenerated.

8. The absorbent fiber of claim 1 wherein the cellulose fiber is regenerated.

9. The absorbent fiber of claim 1 wherein the absorbent fiber has the appearance of an unmodified cellulose fiber.

10. The absorbent fiber of claim 1 wherein the absorbent fiber has the appearance of a regenerated cellulose fiber.

11. An absorbent article comprising:
a topsheet;
a backsheet; and
an absorbent core disposed between the topsheet and the backsheet;
wherein the absorbent core includes superabsorbent material and absorbent fibers;
wherein the absorbent fibers comprise cellulose fibers that have been modified via chemical modification;
wherein the chemical modification is selected from carboxymethylation, sulfonation, sulfation or phosphonation; and
wherein the modified fibers exhibit a Centrifuge Retention Capacity after four hours of between 1.4 and 8 grams per gram and an absorption rate between 0.0001 and 0.01 sec⁻¹.

12. The absorbent article of claim 11 wherein the absorbent fibers further comprise a crosslinking agent.

13. The absorbent article of claim 11 wherein the absorbent fibers have a degree of substitution of less than 0.5.

14. The absorbent article of claim 11 wherein the absorbent fibers have a degree of substitution of greater than 0.5.

15. The absorbent article of claim 11 wherein the absorbent fibers have an average degree of carboxyl group substitution of from about 0.1 to about 0.7.

16. The absorbent article of claim 11 wherein the absorbent fibers further comprise a reagent.

17. The absorbent article of claim 11 wherein the cellulose fibers are non-regenerated.

18. The absorbent article of claim 11 wherein the cellulose fibers are regenerated.

19. The absorbent article of claim 11 wherein the absorbent fibers have the appearance of unmodified cellulose fibers.

20. The absorbent article of claim 11 wherein the absorbent fibers have the appearance of regenerated cellulose fibers.

21. The absorbent article of claim 11 wherein the absorbent core comprises at least about 10% by weight of the absorbent fibers.

22. The absorbent article of claim 11 wherein the absorbent core comprises between 10% and 70% by weight of the absorbent fibers.

23. The absorbent article of claim 11 wherein the absorbent core comprises at least about 30% by weight of the superabsorbent material.

24. The absorbent article of claim 11 wherein the absorbent core comprises about 60% to about 95% by weight of the superabsorbent material.

25. The absorbent article of claim 11 wherein the absorbent core further comprises fluff.

26. The absorbent article of claim 11 wherein the absorbent core further comprises a surfactant.

27. The absorbent article of claim 11 wherein the article is selected from personal care absorbent articles, health/medical absorbent articles, household/industrial absorbent articles and sports/construction absorbent articles.
